(19) United States
(12) Patent Application Publication
(10) Pub. No.: US 2020/0029925 A1
(43) Pub. Date: Jan. 30, 2020

(54) MYOCARDIAL BLOOD FLOW WITH RELIABILITY FEATURE

(71) Applicants: Siemens Medical Solutions USA, Inc., Malvern, PA (US); University of Oxford, Oxford (GB)

(72) Inventors: Antoine Saillant, Knoxville, TN (US); Michael Chappell, Oxford (GB); Vijay Shah, Knoxville, TN (US)

(21) Appl. No.: 16/047,381

(22) Filed: Jul. 27, 2018

(51) Int. Cl. A61B 6/00; A61B 6/03; G06K 9/62

(52) U.S. Cl. CPC A61B 6/507; A61B 6/037; A61B 6/503; G06K 9/6296; A61B 6/5264

(57) ABSTRACT

A method comprises: scanning a patient with a PET scanner; computing a patient myocardial blood flow (MBF) parameter value and a patient MBF variation value of the patient based on the scanning; comparing the patient MBF variation value to an MBF variation threshold; and determining that the patient MBF parameter value is unreliable in response to determining that the patient MBF variation value is greater than the MBF variation threshold.

### FIELD

This disclosure relates generally to medical imaging, and more specifically to methods and apparatus for estimation of myocardial blood flow.

### BACKGROUND

Quantification of myocardial blood flow (MBF) is important for dynamic cardiac positron emission tomography (PET) imaging. By comparing MBF values between a resting state and after a response to physiological or pharmacological stress, clinicians can also evaluate Myocardial Flow Reserve (MFR), which is the ratio of stress MBF and rest MBF. Together, MBF and MFR can improve diagnosis, for example in patients with heart disease.

The estimation of MBF in dynamic PET can be biased by many different processes, such as the choice of reconstruction method, the type of tracer, or the statistical noise of the scanner. MBF estimates can also be affected by temporal sampling strategy, post-processing methods, spillover from the right ventricle (RV) into the interventricular septum, and patient motion. Patient motion can be a major source of error, particularly in clinical applications. With patient motion, the region studied may not reflect the same tissue over different image frames, and consequently the final estimate of MBF is less reliable.

Reliability of MBF has been assessed with a visual confirmation of the perfusion image and a review of the dynamic scan. Often a careful visual inspection of the Myocardial Perfusion Image (MPI) has been performed to validate MBF parameters. This solution is however subjective, and depends on the reviewing clinician's experience.

### SUMMARY

In some embodiments, a method comprises: scanning a patient with a PET scanner; computing a patient myocardial blood flow (MBF) parameter value and a patient MBF variation value of the patient based on the scanning; comparing the patient MBF variation value to an MBF variation threshold; and determining that the patient MBF parameter value is unreliable in response to determining that the patient MBF variation value is greater than the MBF variation threshold.

In some embodiments, a method comprises: accessing a plurality of verified positron emission tomography (PET) image datasets; fitting a myocardial blood flow (MBF) model to each of the plurality of verified PET image datasets, to determine at least one MBF parameter value and at least one variation value for each respective one of the plurality of PET image datasets; determining a distribution of the variation among the plurality of datasets; determining an MBF variation threshold for the at least one MBF parameter based on the distribution; scanning a patient with a PET scanner; computing a patient MBF parameter value and a patient MBF variation value of the patient based on the scanning; and determining that the patient MBF parameter value is unreliable in response to determining that the patient MBF variation value is greater than the MBF variation threshold.

In some embodiments, a system comprises: a scanner capable of detecting activity of a tracer in a patient; a processor communicatively coupled to the scanner; and a non-transitory, machine readable storage medium storing instructions and data. The data comprise a myocardial blood flow (MBF) variation threshold. The instructions configure the processor to perform a method comprising: receiving data from scanning a patient using the scanner; computing a patient MBF parameter value and a patient MBF variation value of the patient based on the scanning; comparing the patient MBF variation value to the MBF variation threshold; and determining that the patient MBF parameter value is unreliable in response to determining that the patient MBF variation value is greater than the MBF variation threshold.

### BRIEF DESCRIPTION OF THE DRAWINGS

FIG. 1 is a schematic diagram of an exemplary positron emission tomography (PET) system

FIG. 2 is a block diagram showing instruction components of the system in FIG. 1.

FIG. 3 is a block diagram of a one-compartment model of a kinetic model.

FIG. 4 is a graph showing tracer concentration data and a model fit to the data.

FIG. 5 shows a distribution of the K₁ myocardial blood flow (MBF) parameter.

FIGS. 6A-6C show coefficient of variation (CV) data for the K₁ parameter for the left anterior descending artery (LAD), left circumflex (LCX), and right coronary artery (RCA), respectively.

FIGS. 7A-7C are PET images of the heart, including the short axis view, horizontal long axis view, and vertical long axis view, respectively.

FIG. 8A is a polar map of MBF in the LCX, RCA, and LAD.

FIG. 8B is a polar map of the CV of the MBF data in FIG. 8A.

FIG. 8C is a simplified polar map of the CV of the MBF data in FIG. 8A, for providing “go, no-go” information.

FIG. 9 is a flow chart of a method for MBF reliability determination.

### DETAILED DESCRIPTION

This description of the exemplary embodiments is intended to be read in connection with the accompanying drawings, which are to be considered part of the entire written description.

Quantification and standardization of Myocardial Blood Flow (MBF) estimation with dynamic positron emission tomography (PET) is an important area of research for dynamic cardiac PET imaging. A variety of factors (e.g., image reconstruction protocols or post-processing methods) may influence the estimates. For the kinetic modelling aspect itself the partial volume effect (PVE) between the left ventricle cavity and the myocardial tissue may be challenging to overcome. In cases with low myocardial perfusion, the averaged signal obtained from the tissue is dominated by the cavity blood signal, which can lead to erroneous MBF estimates.

This disclosure describes systems and methods that estimate MBF parameters (e.g., K₁; k₂; Vb) along with a quantitative value representing the uncertainty in the estimate during dynamic cardiac PET imaging. As used herein, K₁ represents the exchange rate of a material (e.g., tracer) from the blood to the tissue compartment, k₂ represents the transfer of a material from the tissue compartment to the blood, and V_{b} represents percentage of arterial blood vessel within the tissue, defined between 0 and 1.

In some embodiments, the system and method determine an uncertainty measure for an MBF parameter estimate from new scan data in real-time or near real-time. If the uncertainty is above a predetermined threshold, motion correction is applied to the PET image data, and the image is re-processed through the image processing chain. The MBF parameter(s) and uncertainty are again computed in real-time, based on the motion-corrected image. If the uncertainty value still exceeds the threshold, the system alerts the clinician to repeat the scan, because the original MBF parameter estimate is considered unreliable.

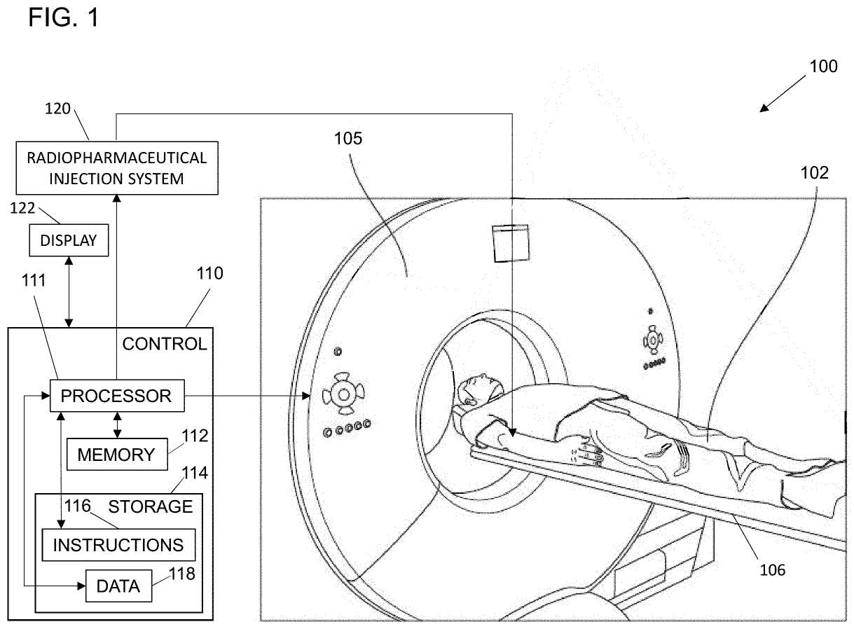

FIG. 1 shows a scanner system 100, including a control device 110 for controlling a scanner 105. The scanner 105 can be an magnetic resonance (MR) scanner, such as a “MAGNETOM VIDA”™ scanner, a computed tomography (CT) scanner, such as a “SOMATOM CONFIDENCE RT Pro”™ CT Scanner, a PET scanner, such as the “BIOGRAPH HORIZON”™ PET/CT scanner, “SYMBIA INTEVO”™ Single-photon emission computed tomography (SPECT)/CT system, or an ultrasound scanner, such as the “ACUSON SC2000PRIME”™ cardiovascular ultrasound system, all sold by Siemens Medical Solutions USA, Inc. of Malvern, Pa. The scanner can include an automated contrast agent injection system 120 for automatic control of the injection profile, as provided by “CARE CONTRAST”™ in the “SOMATOM”™ scanner by Siemens Medical Solutions USA, Inc. of Malvern, Pa., where the contrast injector can be connected to the CT scanner, enabling synchronized injection and scanning. These are only examples, and other scanner makes and models may be used.

In some embodiments, the scanner 105 can be a continuous bed motion scanner, capable of moving a bed 106 of the scanner from a beginning of the one or more scans to an end of the one or more scans. The scanner 105 has a movable bed for receiving a patient and a plurality of detectors (not shown) for detecting a radiopharmaceutical in a blood vessel of the patient. Either the bed 106 or the plurality of detectors (not shown) are movable. In other embodiments, the scanner is capable of step-and-shoot scanning, with the sampling being performed at each of the two or more locations while the bed is not moving.

As discussed herein, a “scan” or “pass” can refer to a single translation by the scanner bed 106 with respect to the scanner 105, or a single translation by the scanner 105 with respect to the scanner bed 106. A scan or pass can proceed in a head-to-toe direction (corresponding to the bed moving in a direction from the patient's feet toward the patient's head), or a toe-to-head direction (corresponding to the bed moving in a direction from the patient's head toward the patient's feet). A scan or pass can refer to a complete pass (in which the patient's body from head to feet passes the scanner 105), or a partial pass (in which only a portion (less than 100%) of the patient's body (e.g., the patient's heart) passes the scanner 105). As used herein, the terms “scan” and “pass” can have any combination of these three attributes.

The control device 110 has a processor 111 configured to cause the scanner 105 to perform one or more scans of the patient and detect emissions indicative of presence of a the radiopharmaceutical in a the blood vessel of the patent. Each of the one or more scans includes estimating the MBF based on the concentration of the radiopharmaceutical or contrast material, for modeling MBF based on the estimation.

The processor 111 is configured (e.g., by software) for controlling the scanner 105 based on the estimated MBF, injection profile, and delay between injecting the radiopharmaceutical or contrast agent and performing the scan. The processor 111 can issue commands to the automated injection system 120, to inject a selected dosage of radiopharmaceutical or contrast material in accordance with the estimated AIF. The processor 111 can have user input/output devices, such as a display 122, which can be a touch-screen capable of receiving user inputs and displaying outputs. Other input devices (e.g., keyboard or pointing device, not shown) may be included.

The processor 111 can include an embedded processor, a computer, a microcontroller, an application specific integrated circuit (ASIC), a programmable gate array, or the like. The control device 110 includes a main memory 112, which can include a non-transitory, machine readable storage medium such as dynamic random access memory (DRAM). The secondary memory comprises a non-transitory, machine readable storage medium 114, such as a solid-state drive, hard disk drive (HDD) and/or removable storage drive, which can include a solid state memory, an optical disk drive, a flash drive, a magnetic tape drive, or the like. The non-transitory, machine readable storage medium 114 can include tangibly store therein computer software instructions 116 for causing the scanner 105 to perform various operations (described herein) and data 118.

The injection system 120 can perform calibrated injections to patients, starting from a multi-dose solution of fluorodeoxyglucose (FDG), iodine, or other radiopharmaceuticals, or a contrast material. In some embodiments, the scanner 100 is not equipped with an automated injection system 120, in which case a separate injection system (not shown) may be used. For example, some systems can include an external injection system (not shown), such as the “IRIS™” Radiopharmaceutical Multidose Injector sold by Comecer S.p.A. of Castel Bolognese, Italy. In some embodiments, the injection system 120 has a wired or wireless communications link with the processor 111, for automatically transmitting dosage, injection protocol and scan delay to the injection system 120.

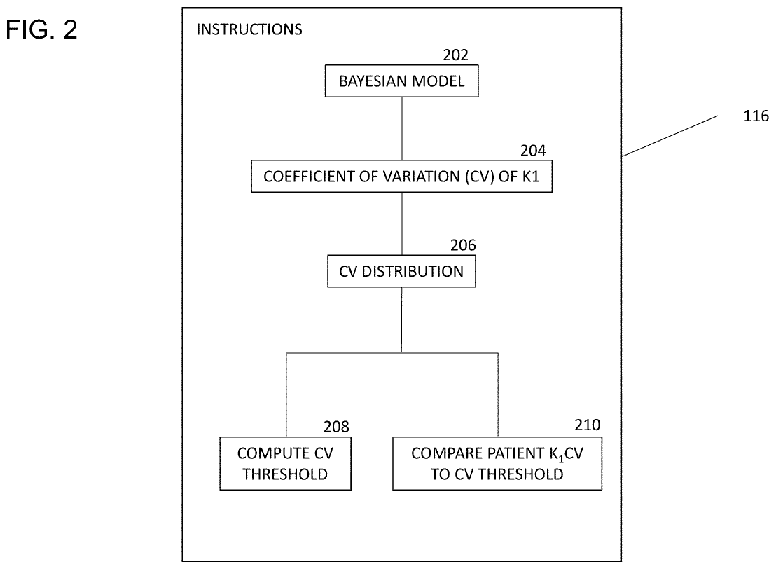

FIG. 2 is a block diagram of a portion of the instructions block 116 of FIG. 1.

Some embodiments use a Bayesian framework, representing the kinetic parameters as a probability distribution, to model myocardial blood flow (MBF). In addition to estimating the kinetic MBF parameters, Bayes theorem also offers a framework to estimate uncertainties of parameters in a model. The Bayesian framework provides uncertainty measures for one or more of the kinetic parameters. If the extracted uncertainty is high, the parameter studied is considered to have high variability—or low confidence. Blocks 202-208 are performed while populating the system database (discussed below), and block 210 is performed in the clinical setting.

A Bayesian model block 202 uses the time-activity-curve (TAC) data from a patient scan dataset to obtain a set of MBF parameter values. For example, the Bayesian model can estimate K₁, k₂, and V_{b} for each patient based on the respective patient scan dataset.

In a Bayesian framework, each parameter is represented as a probability distribution instead of a single value, from which an uncertainty metric can be drawn. Block 202 can use various methods to solve the Bayes inference problem, such as Monte Carlo Markov Chain (MCMC), a family of sampling algorithms. Another way to perform Bayesian inference is to use Variational Bayes (VB), a fully Bayesian approach that uses variational theory to approximate the solution to the posterior distribution that is the output of a Bayesian analysis. VB has lower computational cost than MCMC.

A block 204 normalizes the MBF parameter values from block 202. In some embodiments, for purpose of assessing the reliability of the MBF estimate, the reliability of a single MBF parameter is assessed to determine whether to apply motion correction and/or repeat the scan. For example, in some embodiments, the MBF parameter K₁ is used as a surrogate for MBF during reliability assessment. In some embodiments, the respective standard deviation σ for the value of K₁ from the Bayesian model for each respective patient represents reliability. In some embodiments, the standard deviation can be normalized by computing a coefficient of variation (CV=σ/K₁) for each patient, as a dimensionless value indicating reliability of a K₁ estimate relative to K₁ for each patient.

Block 206 constructs a database of a priori (simulated or clinical) MBF confidence data used to determine a probability distribution function for the coefficient of variation (CV=σ/μ) of K₁. The respective CV value for each patient provides a respective data point in the database. In some embodiments, patient scan CV data points are only included in the database if the images reconstructed from the patient scan have been verified (validated) as acceptable by an expert or clinician. Assuming a Gaussian distribution of CV values for validated images, 95% of the CV values are within 1.96 standard deviations of the mean CV value.

At block 208, a threshold CV value is selected (e.g., the mean CV±1.96σ or 2σ). A patient scan dataset having a CV value at or below the threshold can be considered acceptable, and a patient scan dataset having a CV value above the threshold can be considered unreliable.

Block 210 applies the CV threshold to evaluate the reliability of new scans in a clinical setting. When a new scan is completed, Bayesian model fitting is performed, providing an estimated mean value (μ_{K1}) of K₁ and the standard deviation σ_{K1} (or variance σ_{K1}²) of K₁ for the new scan. The method then determines whether the CV (=σ_{K1}μ_{K1}) of the estimated K₁ for the new scan exceeds the threshold CV (e.g., μ±2σ) computed by block 208. If the CV of the estimated K₁ exceeds the threshold CV, motion correction is applied to the image data, the PET image is re-processed, the Bayesian model is again fitted, and K₁ is again computed. If the CV of K₁ is still outside the acceptable range, the scan is repeated.

In some embodiments, clinical datasets may be corrected for motion, and the MBF uncertainties may be compared before and after motion correction to determine whether to repeat the scan. Based on training data comprising uncertainty estimates from normal cases and abnormal scans (for which MBF values could be misleading), the system and method can be used to automatically flag unreliable scans and instruct the clinician to repeat the scan for which an unreliable dataset was obtained.

MBF Estimation and PET Compartmental Models

Computation of MBF includes kinetic modelling of Time Activity Curves (TACs) of the myocardial tissue. TACs represent the evolution of the tracer as a function of time, and can be described by a kinetic model, from which parameters are then subsequently used to compute the MBF.

To compute MBF, TACs are derived from two Regions of Interest (ROIs): the left ventricular (LV) cavity to obtain the arterial blood and the myocardial tissue.

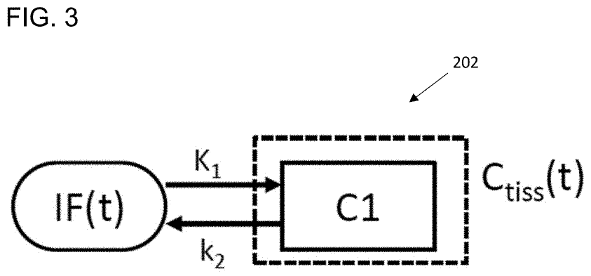

In PET kinetic modelling, compartmental models describe the uptake of the tracer in the tissue. Each compartment of the model represents a possible state of the tracer, specifically its physical location or its chemical form as shown in FIG. 3.

FIG. 3 is a block diagram of an exemplary single-compartment model for the Bayesian model 202. In FIG. 3, blood is indicated by IF(t), and the tissue compartment is shown as C1. In some embodiments, the model depends on the tracer used and the type of the tissue studied. In some embodiments, for the tracer Rubidium-82 (Rb-82) used for MBF quantification, the model is a one-compartment tissue model. The response function R(t) modelling the tracer exchange between the tissue and the blood is described in equation (1):

R(t)=K₁ exp(−k₂t)*IF(t) (1)

where: IF(t) is the time course of the concentration of the tracer in arterial blood, K₁ and k₂ are the two exchange rates between the blood and the tissue, and * is the convolution operator. In a one-compartment model, K₁ is the constant for tracer from the blood entering the tissue, and k₂ is the constant for tracer leaving the tissue to enter the blood.

In addition, a partial volume effect correction from the arterial blood is applied in equation (2). Substituting equation (1) into equation (2) yields equation (3):

Cₜᵢₛₛ(t)=(1−V_{b})·R(t)+V_{b}·IF(t) (2)

Cₜᵢₛₛ(t)=(1−V_{b})·[K₁ exp(−k₂t)*IF(t)]+V_{b}IF(t) (3)

where: Vb is the percentage of arterial blood vessel within the tissue, defined between 0 and 1.

In order to obtain the MBF for Rb-82 images, the Renkin-Crone equation is applied, because of non-linearity in the relationship between K₁ and MBF:

K₁=MBF[1−A exp(−B/MBF)] (4)

where: A and B are defined between 0 and 1, and take different values depending on the tracer properties. The form of Equation (4) shows that K₁ is an increasing function of MBF. Therefore a reduction of uncertainty in K₁ is reflected by a reduction of uncertainty in MBF. Thus, in determining the acceptability of a PET scan dataset (with or without motion correction) the kinetic parameter K₁ can be used as a surrogate for MBF. Because the computation of K₁ is faster than the computation of MBF, K₁ can be computed in real time to determine whether to apply motion correction and/or repeat a scan.

Measurement of Uncertainty with Variational Bayes

A PET kinetic model M is parameterized with a set of N parameters p={p₁, . . . , p_{N}}. The measured signal over the M time points is denoted y={y₁, . . . , y_{M}}. In a non-limiting example where N=3, the parameters are (K₁; k₂; V_{b}), and y is a time-activity curve (TAC) derived from the myocardial tissue. Assuming that the noise on the signal is additive Gaussian noise with precision ϕ, one can define θ={p,ϕ} as the full set of parameters for the generative model of the data. The PET kinetic model f(t,p)=Cₜᵢₛₛ(t), (see equation (3)), estimates y={f(tⱼ)}_{j∈1,m}) with the most probable parameters p.

Using Bayes theorem, the posterior probability distribution for the model parameters can be estimated given the data y:

$$P ( θ \vert y ) = \frac{P ( y \vert θ ) P ( θ )}{P ( y )} \qquad ( 5 )$$

Where:

The prior P(θ) is the distribution on the parameters capturing prior knowledge of their value before any new data has been considered. The likelihood P (y|θ)is the probability of observing y given a set of parameters θ, and is computed directly from the model and the observation of the data, the TAC y in the case of PET kinetics. The evidence P(y) is the distribution of the observed data, marginalized over the parameters θ, P(y)=∫P(y|θ)P(θ)dθ.

In some embodiments MCMC algorithms can be used to solve the equations arising from Bayes theorem by sampling the posterior distribution through the construction of a Markov Chain that converges to the posterior distribution after a certain number of iterations.

In some embodiments, Variational Bayes (VB) can be used to approximate the posterior distribution. VB has fast convergence and comparatively inexpensive computations. To solve Bayes equations, VB approximates the true posterior P(θ|y) with a simpler form Q(θ). Solving the equations from Bayes theorem to provide the posterior distribution is then reduced to the maximization of the free energy F, as defined in equation (6):

$$F = \int Q ( θ ) log ( \frac{P ( y \vert θ ) P ( θ )}{Q ( θ )} ) d θ \qquad ( 6 )$$

The distribution Q(θ) can be chosen using the mean field approximation, for the kinetic modelling application the parameters of the kinetic model p and the noise model ϕ are considered to be independent:

Q(θ)=Qₚ(p|y)Q_{ϕ}(ϕ|y)

In some embodiments, the priors chosen for the application of the VB algorithm in PET kinetic modelling may be a multivariate Normal (MVN) for the kinetic model parameters and a Gamma distribution Ga for the noise precision ϕ as in equations (7) and (8), respectively.

P(p)=MVN(p, m,Σ⁻¹) (7)

P(ϕ)=Ga(ϕ, s, c) (8)

Each kinetic parameter is thus represented by a marginal distribution on pᵢ, i ∈ {1, . . . , N} which follows a normal distribution N(μᵢ; σᵢ²) of respective mean and standard deviation (μᵢ; σᵢ). The mean μᵢ may be taken as the best estimate for the parameter pᵢ, while the standard deviation σᵢ is associated with the uncertainty in the measurement.

Similarly, one can draw intervals for the parameters; for example, if a parameter pᵢ has a Gaussian distribution, the 95% confidence interval CI₉₅ of pᵢ is defined by equation (9):

CI₉₅(p)=[μᵢ−1.96σᵢ; μᵢ+1.96 σᵢ] (9)

The wider this interval the more uncertain is the estimated value of pᵢ. Another way to look at the uncertainty is to compute the coefficient of variation CV(pᵢ) according to equation (10):

CV(pᵢ)=σ₁/μᵢ; (10)

The coefficient of variation represents a unitless metric that allows comparison across different datasets. Similarly the higher CV is, the higher the uncertainty on the parameter becomes.

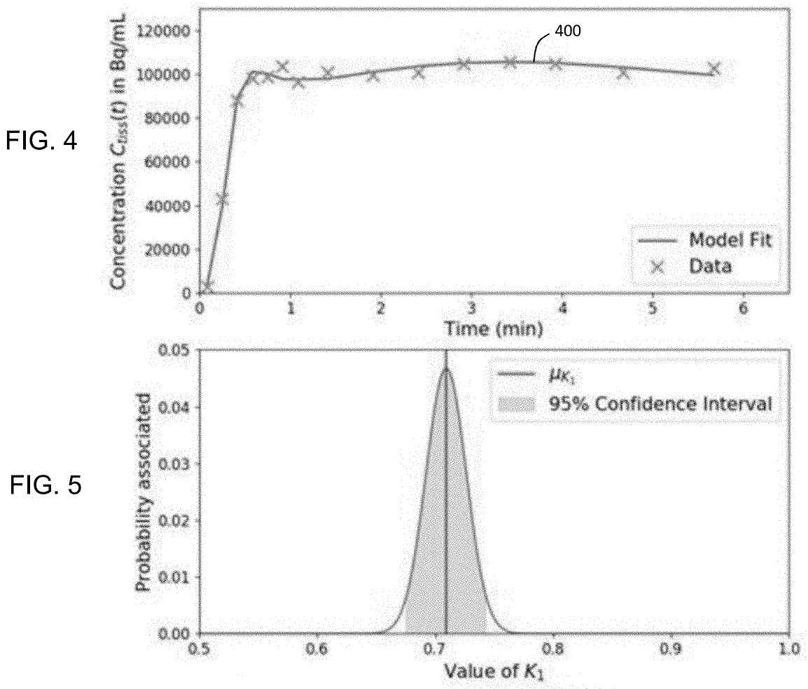

FIG. 4 is a diagram showing an example of the concentration Cₜᵢₛₛ(t) of tracer in the first six minutes after injection for a patient. FIG. 4 shows the individual concentration data points from the scan (indicated by “X”), and the corresponding Bayesian model curve 400. The model also provides an uncertainty measure σ.

FIG. 5 shows an example of the probability distribution of the coefficient of variation of K₁ for a single patient. The coefficient of variation of K₁ can be estimated by a Gaussian distribution function with a mean value μ_{K1} and a standard deviation σ_{K1} (where σ comes from the normal distribution of K₁. The variation of FIG. 5 can be normalized by determining the CV(K₁)=(σ_{K1}/μ_{K1}). Each patient scan dataset in the database has a corresponding CV value.

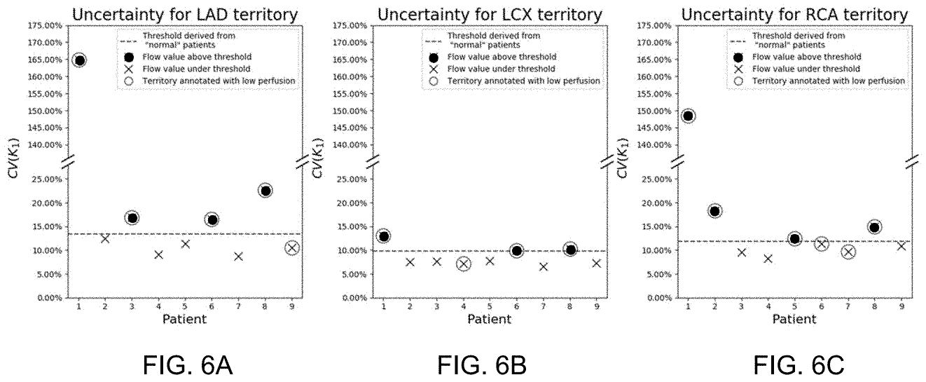

FIGS. 6A-6C show results of an example of the use of MBF reliability data. A cohort of 18 Rubidium stress scans were examined, equally split between patients with visually normal and low myocardial perfusion. MBF values of the normal patient cohort were reviewed by an independent expert and considered as plausible representation of blood flow. Within the 9 abnormal datasets, 15 territories with reduced perfusion were considered (5 for left anterior descending artery (LAD), 4 for left circumflex (LCX), 6 for right coronary artery (RCA)). The blood input function (BIF) and the time activity curves (TACs) were extracted using “SYNGO.VIA” software from Siemens Medical Solutions USA, Inc., Malvern, Pa.

TACs were fitted with a one compartment model, with spillover factor (SF), using a Variational Bayes (VB) algorithm for nonlinear model fitting. Each parameter (K₁, k₂ and SF) was represented by a Normal distribution N(μ,σ²), where μ was taken as the best estimated parameter value, and σ as a measure of reliability in the estimate. For purpose of evaluating reliability, K₁ was used as a surrogate for MBF. The coefficient of variation CV(K₁)=σ/μ was calculated for each patient (FIGS. 6A-6C) and is used as an unitless measure of the reliability of the flow estimate. The mean and standard deviation of CV for the healthy patient cohort was computed, and the threshold defined by μ+2σ was considered as the upper limit above which the K₁ estimate could be considered unreliable.

FIGS. 6A-6C show the values of CV(K₁) for the 9 disease patients. Out of the 15 territories annotated by circular outline (O) as low perfusion, 11 K₁ values were categorized as unreliable according to the method. These 11 K₁ values are indicated in FIGS. 6A-6C by a solid dot within a circular outline. In addition, none of the values indicating acceptable perfusion were classified as unreliable. That is, as shown in FIGS. 6A-6C. all of the values having CV(K₁) values above the threshold (dashed line) were also classified as unreliable by the independent expert. The average values of CV were 9.9% for LAD, 6.5% for LCX and 8.0% for RCA. The threshold derived was 13.3% for LAD, 9.8% for LCX and 11.8% for RCA. Thus, scan results for which the CV of K₁ is outside the μ±2σ threshold are strongly indicated as being unreliable. Thus, using the method, the scans for which at least one territory in which, the K1 values were determined to be unreliable could be flagged in real-time or near real-time, to have the scans repeated.

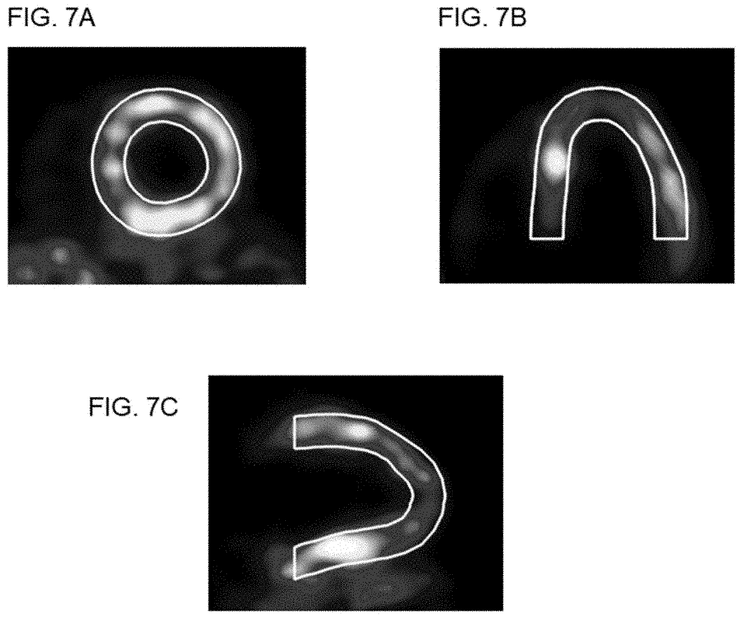

FIG. 7A-7C show standard PET image views of a patient's heart, including a short axis view (in FIG. 7A), a horizontal long axis view (in FIG. 7B), and a vertical long axis view in FIG. 7C. FIGS. 7A-7C are shown in gray scale format, so that the areas of greatest blood flow are lightest, and areas with smallest blood flow are darkest.

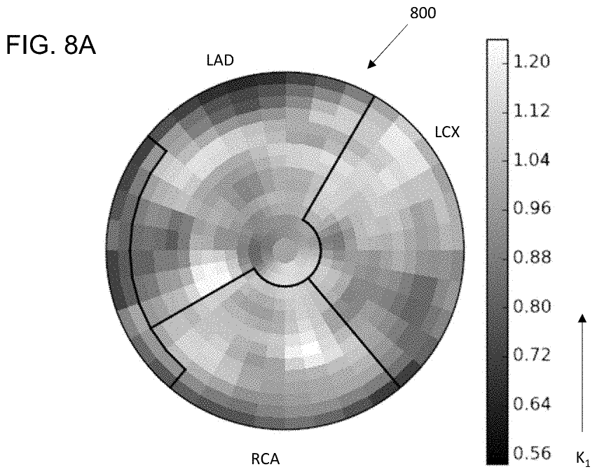

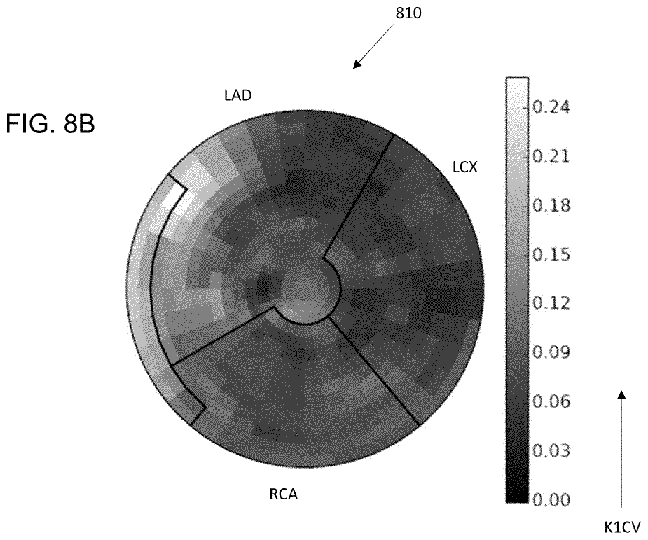

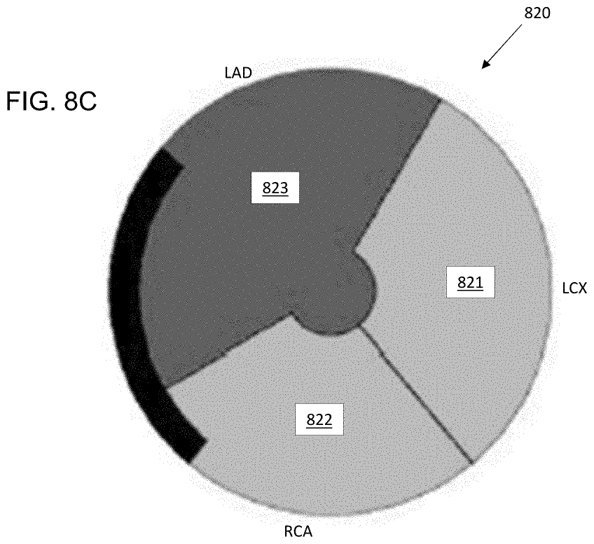

Some embodiments provide a quality control map for MBF values in an easily interpretable format. A model fitting algorithm can estimate and display the MBF parameter values. For example, FIG. 8A displays the K₁ parameter using a polar map format 800, as described in Garcia, E. V., et al., “Quantification of Rotational Thallium-201 Myocardial Tomography,” Journal of Nuclear Medicine, 26(1):17-26. (1985). The polar map can display perfusion in the three-dimensional (3D) cardiac surface in a two-dimensional (2D) format. In the polar map, the eastern portion of the map corresponds to the left circumflex (LCX), the northwestern portion corresponds to the left anterior descending artery (LAD), and the southwestern portion corresponds to the right coronary artery (RCA)).

In some embodiments of this disclosure, in addition to an MBF parameter value, the system also generates a confidence measurement in a polar map 810 (as shown in FIG. 8B). Each segment in the confidence polar map 810 of FIG. 8B identifies a CV corresponding to the estimated MBF value of the corresponding segment in the MBF polar map of FIG. 8A.

In some embodiments, the clinician can look at the CV in each individual zone in the reliability polar map of FIG. 8B, and individually assess relevance of the corresponding MBF data in FIG. 8A.

In other embodiments, as shown in FIG. 8C, the confidence map 820 can be provided in a “go, no-go” summary format. In FIG. 8C, a respective combined reliability measure is computed for each of the regions (also referred to as “territories”) LCX 821, RCA 822 and LAD 823 in FIG. 8C, based on the individual CV values for each smaller zone in FIG. 8B. The CV values in a given territory are computed as the average of the 100+ cells within each 1/3 of the polar map. The combined reliability values of regions 821-823 are compared to the CV threshold produced by block 208 (FIG. 2). Each region having a CV below the CV threshold can be indicated as acceptable (e.g., by green color); each region having a CV of equal to or greater than the CV threshold value can be indicated as unreliable (e.g., by red color). If any of the three regions 821-823 is indicated to be unreliable, then the PET images are reprocessed with motion correction, and if any of the three regions 821-823 is still indicated to be unreliable, then the operator is alerted to repeat the PET scan of the patient.

In some embodiments, the polar map of FIG. 8B or FIG. 8C is displayed on the display device 122 (FIG. 1). In other embodiments, the polar map of FIG. 8B or FIG. 8C is printed on printer (not shown). In some embodiments, the polar map results automatically trigger application of motion correction and re-processing of the images for a scan, as discussed above.

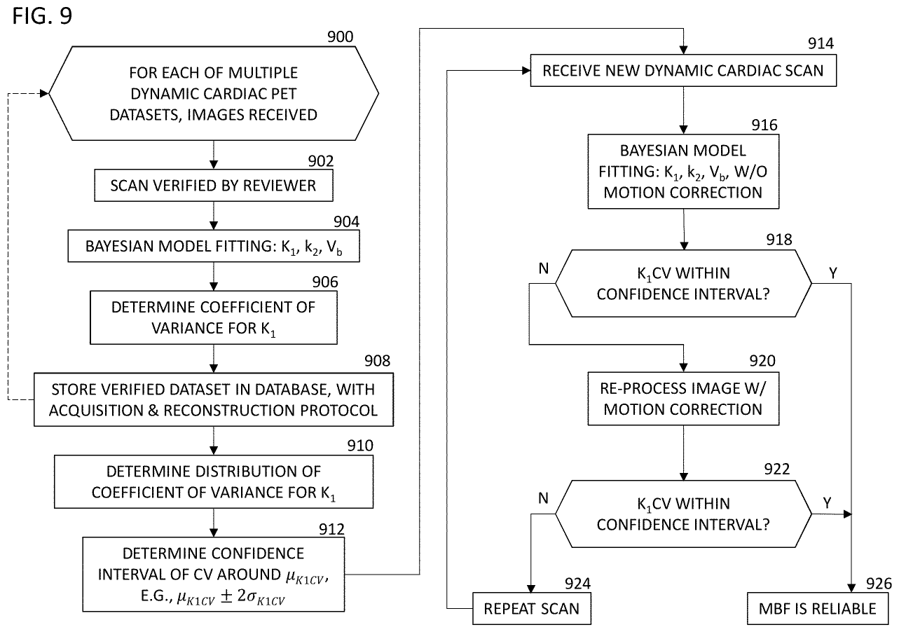

FIG. 9 is a flowchart of an exemplary method. In some embodiments, a two-step workflow includes a training phase (steps 900-912) and a clinical phase (steps 914-926).

At step 900, a loop including steps 902-908 is repeated for each of a plurality of dynamic cardiac PET datasets and images that have been received. Thus, a plurality of patient's hearts are scanned, and images are received and reconstructed.

At step 902, the image reconstructed from the scan is verified by a reviewer. In some embodiments, one or more experts review the images and provide a subjective confidence value characterizing the image on a continuum between acceptable reliability and corrupted. The confidence value is an assessment of the reliability of the processed image (as opposed to being a measure of the health of the patients).

At step 904, a model is fit to the dataset. For example, in some embodiments, Variational Bayes or Monte-Carlo Markov Chain (MCMC) inference are used. In some embodiments, the model estimates K₁, k₂ and V_{b}. In some embodiments, the system provides an MBF parameter (e.g., K₁) for cardiac tissue in a polar graph format, along with corresponding reliability (e.g., CV=σ₁/μ₁) in a polar graph format as shown in FIG. 8B or 8C).

At step 906, the CV of K₁ block 204 of processor 116 determines the CV of K₁ for the patient dataset.

At step 908, the verified dataset is stored in a CV database, along with acquisition protocol information and reconstruction protocol.

At step 910, the distribution of the CV of K₁ is determined. In some embodiments, a separate distribution is determined for each respective acquisition protocol.

At step 912, the processor 116 determines a confidence interval for the CV around the mean CV for K₁ in the training database. For example, in some embodiments, the confidence interval for the CV is given by μ_{K1CV}±2σ_{K1CV}.

At step 914, a new scan of a patient is received in a clinical setting, according to an acquisition protocol.

At step 916, a Bayesian model is fit to the patient scan data without motion correction. For example, the model can estimate K₁, k₂ and V_{b}.

At step 918, a determination is made whether the CV of K₁ for the patient scan is within the confidence interval (i.e., whether CV is less than the CV threshold value corresponding to the acquisition protocol). If the CV is within the confidence interval, step 926 is performed. If the CV is outside the confidence interval, step 920 is performed.

At step 920, the images corresponding to the patient scan dataset are reprocessed with motion correction.

At step 922, the CV is recalculated, and a determination is again made whether the CV of K₁ for the patient scan is within the confidence interval. If the CV is within the confidence interval, step 926 is performed. If the CV is outside the confidence interval, step 924 is performed.

At step 924, in response to determining that—even after motion compensation—the CV for K₁ is outside of the confidence interval for the CV, the scan data are deemed unreliable, and the scan is repeated.

At step 926, the MBF is considered sufficiently reliable for dynamic PET imaging.

Although examples are described above for evaluating reliability of myocardial images, the methods can be extended to other organs. Although examples are described above using single-compartment models, a variety of models can be used. Although examples are described above in which a 95% confidence interval is used for the CV of an MBF parameter, a different confidence interval (e.g., 90%) can be used in other embodiments.

The method and systems described herein can provide automated quality control for PET image scans, and initiate motion compensation and additional processing to improve PET image reliability. The method can flag unreliable scan data and provide an indication when a scan should be repeated.

The methods and system described herein may be at least partially embodied in the form of computer-implemented processes and apparatus for practicing those processes. The disclosed methods may also be at least partially embodied in the form of tangible, non-transitory machine readable storage media encoded with computer program code. The media may include, for example, RAMs, ROMs, CD-ROMs, DVD-ROMs, BD-ROMs, hard disk drives, flash memories, or any other non-transitory machine-readable storage medium, wherein, when the computer program code is loaded into and executed by a computer, the computer becomes an apparatus for practicing the method. The methods may also be at least partially embodied in the form of a computer into which computer program code is loaded and/or executed, such that, the computer becomes a special purpose computer for practicing the methods. When implemented on a general-purpose processor, the computer program code segments configure the processor to create specific logic circuits. The methods may alternatively be at least partially embodied in a digital signal processor formed of application specific integrated circuits for performing the methods.

Although the subject matter has been described in terms of exemplary embodiments, it is not limited thereto. Rather, the appended claims should be construed broadly, to include other variants and embodiments, which may be made by those skilled in the art.

What is claimed is:

1. A method comprising:
accessing a plurality of verified positron emission tomography (PET) image datasets;
fitting a myocardial blood flow (MBF) model to each of the plurality of verified PET image datasets, to determine at least one MBF parameter value and at least one variation value for each respective one of the plurality of PET image datasets;
determining a distribution of the variation among the plurality of datasets;
determining an MBF variation threshold for the at least one MBF parameter based on the distribution;
scanning a patient with a PET scanner;
computing a patient MBF parameter value and a patient MBF variation value of the patient based on the scanning; and
determining that the patient MBF parameter value is unreliable in response to determining that the patient MBF variation value is greater than the MBF variation threshold.

2. The method of claim 1, further comprising applying motion correction to an image data from the scanning to provide a motion corrected image data, in response to determining that the patient MBF parameter value is unreliable.

3. The method of claim 1, further comprising,
computing an updated patient MBF parameter value and an updated patient MBF variation value of the patient based on the motion corrected image data; and
determining that the updated patient MBF parameter value is unreliable in response to determining that the updated patient MBF variation value is greater than the MBF variation threshold.

4. The method of claim 1, wherein the at least one MBF parameter represents an exchange rate of a tracer material from blood to a tissue compartment, the variation value represents a coefficient of variation of the exchange rate, and the MBF variation threshold is a based on a distribution of the coefficient of variation of the exchange rates for the plurality of verified PET image datasets.

5. The method of claim 4, wherein the MBF variation threshold is a mean of the coefficient of variation of the exchange rates for the plurality of verified PET image datasets plus about two times a standard deviation of the coefficient of variation of the exchange rates for the plurality of verified PET image datasets.

6, A method comprising:
scanning a patient with a PET scanner;
computing a patient myocardial blood flow (MBF) parameter value and a patient MBF variation value of the patient based on the scanning;
comparing the patient MBF variation value to an MBF variation threshold; and
determining that the patient MBF parameter value is unreliable in response to determining that the patient MBF variation value is greater than the MBF variation threshold.

7. The method of claim 6, wherein the MBF variation threshold is provided by:
accessing a plurality of verified positron emission tomography (PET) image datasets;
fitting an MBF model to each of the plurality of verified PET image datasets, to determine at least one MBF parameter value and at least one variation value for each respective one of the plurality of PET image datasets;
determining a distribution of the variation among the plurality of datasets;
determining an MBF variation threshold for the at least one MBF parameter based on the distribution; and

8. The method of claim 6, further comprising applying motion correction to an image data from the scanning to provide a motion corrected image data, in response to determining that the patient MBF parameter value is unreliable.

9. The method of claim 6, further comprising,
computing an updated patient MBF parameter value and an updated patient MBF variation value of the patient based on the motion corrected image data; and
determining that the updated patient MBF parameter value is unreliable in response to determining that the updated patient MBF variation value is greater than the MBF variation threshold.

10. The method of claim 6, wherein the at least one MBF parameter represents an exchange rate of a tracer material from blood to a tissue compartment, the variation value represents a coefficient of variation of the exchange rate, and the MBF variation threshold is a based on a distribution of the coefficient of variation of the exchange rates for the plurality of verified PET image datasets.

11. The method of claim 10, wherein the MBF variation threshold is a mean of the coefficient of variation of the exchange rates for the plurality of verified PET image datasets plus about two times a standard deviation of the coefficient of variation of the exchange rates for the plurality of verified PET image datasets.

12. The method of claim 6, wherein computing the patient MBF parameter value includes fitting a Bayesian model to data from the scanning.

13, A system comprising:
a scanner capable of detecting activity of a tracer in a patient;
a processor communicatively coupled to the scanner; and
a non-transitory, machine readable storage medium storing instructions and data, wherein:
the data comprise a myocardial blood flow (MBF) variation threshold; and
the instructions configure the processor to perform a method comprising:
receiving data from scanning a patient using the scanner;
computing a patient MBF parameter value and a patient MBF variation value of the patient based on the scanning;
comparing the patient MBF variation value to the MBF variation threshold; and
determining that the patient MBF parameter value is unreliable in response to determining that the patient MBF variation value is greater than the MBF variation threshold.

14. The system of claim 13, wherein the instructions further comprise instructions for causing the processor to generate the MBF variation threshold by:
accessing a plurality of verified positron emission tomography (PET) image datasets;
fitting a myocardial blood flow (MBF) model to each of the plurality of verified PET image datasets, to determine at least one MBF parameter value and at least one variation value for each respective one of the plurality of PET image datasets;
determining a distribution of the variation among the plurality of datasets;
determining an MBF variation threshold for the at least one MBF parameter based on the distribution; and

15. The system of claim 14, wherein the at least one MBF parameter represents an exchange rate of a tracer material from blood to a tissue compartment, the variation value represents a coefficient of variation of the exchange rate, and the MBF variation threshold is a based on a distribution of the coefficient of variation of the exchange rates for the plurality of verified PET image datasets.

16. The system of claim 15, wherein the MBF variation threshold is a mean of the coefficient of variation of the exchange rates for the plurality of verified PET image datasets plus about two times a standard deviation of the coefficient of variation of the exchange rates for the plurality of verified PET image datasets.

17. The system of claim 13, wherein the instructions further comprise instructions for applying motion correction to an image data from the scanning to provide a motion corrected image data, in response to determining that the patient MBF parameter value is unreliable.

18. The system of claim 13, wherein the instructions further comprise instructions for:
computing an updated patient MBF parameter value and an updated patient MBF variation value of the patient based on the motion corrected image data; and
determining that the updated patient MBF parameter value is unreliable in response to determining that the updated patient MBF variation value is greater than the MBF variation threshold.

19. The system of claim 13, wherein computing the patient MBF parameter value includes fitting a Bayesian model to data from the scanning.
